(19) United States
(12) Patent Application Publication
(10) Pub. No.: US 2021/0378486 A1
(43) Pub. Date: Dec. 9, 2021

(54) SUCTION VALVE FOR AN ENDOSCOPE

(71) Applicant: GA HEALTH COMPANY LIMITED, Shek Mun (HK)

(72) Inventor: Kenneth McCabe, Shek Mun (HK)

(73) Assignee: GA HEALTH COMPANY LIMITED, Shek Mun (HK)

(21) Appl. No.: 16/894,083

(22) Filed: Jun. 5, 2020

(51) Int. Cl. A61B 1/00; A61B 1/015

(52) U.S. Cl. CPC A61B 1/00068; A61B 1/015

(57) ABSTRACT

A suction valve including a valve body having a first portion and second portion including a sidewall extending from the first portion along a valve body longitudinal axis defining a valve body interior. The second portion has an open end forming an inlet and the side wall having an outlet. A stem is disposed in the valve body interior and operably connected to a sealing member. The sealing member has a valve seal disposed thereon. The valve seal is movable between a closed position wherein the valve seal seals the inlet and an open position wherein the inlet is unsealed. The sealing member has a flexible member extending from the stem, wherein the sealing member is angularly deflectable relative to the valve body longitudinal axis when in the open position upon operation of a fluid flowing through the inlet and the outlet.

### FIELD OF TECHNOLOGY

The present invention relates generally to valves for use with medical instruments. More particularly, the present invention relates to a disposable suction valve assembly for use in an endoscopic device.

### BACKGROUND

Endoscopes are used in modern medical practices to allow a medical practitioner to look inside a hollow organ or body cavity of a patient. Using an endoscope, a patient's symptoms may be investigated (e.g., symptoms in the digestive system including nausea, vomiting, abdominal pain, difficulty swallowing, gastrointestinal bleeding, etc.); a diagnosis may be confirmed (e.g., by performing a biopsy); or treatment may be provided (e.g., cauterizing a bleeding vessel, dilating a narrow esophagus, removing a polyp, etc.).

Unlike other medical imaging devices, endoscopes are inserted directly into the organ or cavity. During an endoscopic procedure, air or CO₂ are typically used to insufflate the organ or cavity being accessed and water is used to clean the objective lens of the endoscope. The flow of air and water in and out of the patient is typically controlled by the user of the device via disposable or reusable valves.

Endoscopes are well-known in the art and are commonly used for numerous medical procedures. A control section of an endoscope may include a main body having a suction bore, an air/water bore, and the like. Valves may be inserted into these bores to control various functions of the endoscope. For example, a suction valve for an endoscope may be inserted into a suction bore of the body of the endoscope to provide suction to the endoscope. When the suction valve is in a normal position, air flow from the distal tip of the endoscope is blocked by the valve. When suction is desired, an operator engages the suction valve (e.g. by depressing the valve) to open a suction channel to create negative pressure that draws air or fluid into the opening of an instrument channel of the endoscope. When the operator releases the suction valve, the valve returns to its normal position blocking air flow and stops the suctioning.

After each use, an endoscope may undergo cleaning, disinfection, sterilization, and the like to prevent the spread of disease, germs, bacteria, illness, and the like. Many components of an endoscope may be reusable, such as a suction valve, and must also be cleaned, disinfected, and/or sterilized between uses. Unfortunately, there is usually a great expense associated with maintaining high-level disinfection or sterility of the equipment. Additionally, there exists significant difficulty for access to the suction valve features to properly clean/high level disinfect or sterilize the device.

Reusable suction valves may be assembled from the combination of several metal, plastic, and/or rubber components. As such, there is significant cost associated with the manufacturing of reusable suction valves.

Disposable suction valves obviate the need for cleaning, disinfection, and sterilization, thereby eliminating the cost of repeated cleaning, disinfection, and sterilization. Additionally, disposable suction valves do not require expensive materials to be utilized to manufacture the valves, thereby eliminating the high cost of manufacturing suction valves from expensive materials.

Endoscopes are also used during procedures to collect biopsied tissue samples, such as polyps, to permit their pathology to be determined. Biopsied tissue that is extracted passes into the endoscope and past the suction valve. Removal of these tissue samples can interfere with the valve and negatively affect its performance. In the event that the valve become clogged or otherwise inoperable during a procedure, valuable time must be expended to clear or replace the valve. In addition, removing the valve during the procedure may expose the operator and room personal to a patient's potentially infections effluent solution. Practitioners, may need to section samples to reduce their size in order to allow collection without sticking, clogging or obstructing the valve. However, this process increases the complexity of the procedure.

Accordingly, it would be desirable to provide a suction valve that operates without sticking or clogging and that permits the collection of a wide range of tissue sample sizes without obstructing the valve.

### SUMMARY

The present disclosure provides a suction valve including a valve body having a first portion and second portion including a sidewall extending from the first portion along a valve body longitudinal axis defining a valve body interior. The second portion has an open end forming an inlet and the side wall having an outlet. A stem is disposed in the valve body interior and operably connected to a sealing member. The sealing member has a valve seal disposed thereon. The valve seal is movable between a closed position wherein the valve seal seals the inlet and an open position wherein the inlet is unsealed. The sealing member has a flexible member extending from the stem. The sealing member is angularly deflectable relative to the valve body longitudinal axis when in the open position upon operation of a fluid flowing through the inlet and the outlet.

The present disclosure also provides a suction valve insertable in an endoscopic device having a main body including a bore defined by a bore wall, the bore in communication with a suction pump and an insertion tube, the section valve being disposed in the bore. The suction valve includes a valve body having a first portion and second portion including a sidewall extending from the first portion along a valve body longitudinal axis defining a chamber. The second portion has an open end forming a inlet and the side wall having an outlet. A stem is disposed in the valve body and operably connected to a sealing member. The sealing member has a valve seal disposed thereon. The valve seal is movable between a closed position wherein the valve seal seals the inlet and an open position wherein the inlet is unsealed. The sealing member has a flexible member extending from the stem. The sealing member is deflectable relative to the valve body longitudinal axis toward the bore wall when in the open position upon operation of a fluid flowing through the inlet and the outlet.

The present disclosure still further provides a method of controlling the suction of an endoscope including, obtaining an endoscope including a suction valve. The suction valve includes a valve body having a first portion and second portion including a sidewall extending from the first portion along a valve body longitudinal axis defining a chamber. The second portion has an open end forming an inlet and the side wall having an outlet. A stem is disposed in the valve body and operably connected to a sealing member. The sealing member has a valve seal disposed thereon. The valve seal is movable between a closed position wherein the valve seal seals the inlet and an open position wherein the inlet is unsealed. An actuator is operably connected to the stem. The sealing member has a flexible member extending from the stem. The sealing member is deflectable relative to the valve body longitudinal axis toward the bore wall when in the open position upon operation of a fluid flowing through the inlet and the outlet. The method further includes actuating the actuator to cause the sealing member to move the open position to permit fluid to flow through the suction valve and deactivating the actuator to cause the sealing member to return to the closed position to restrict a flow of fluid through the suction valve.

### BRIEF DESCRIPTION OF THE DRAWINGS

FIG. 1 is a perspective view of an endoscope incorporating a suction valve of the present invention.

FIG. 2 is a perspective view of the suction valve of the present invention.

FIG. 3 is an exploded view of the suction valve.

FIG. 4 is a side view of the suction valve shown in the open position.

FIG. 5 is a sectional view of the suction valve shown in the closed position and disposed in the endoscope.

FIG. 6 is a sectional view of the suction valve shown in the open position and disposed in the endoscope.

FIG. 7 is a sectional view of the suction valve of FIG. 6 showing a flow through the suction valve.

### DETAILED DESCRIPTION

The present disclosure will be described with reference to the accompanying drawings, where applicable. It is understood that the present disclosure may be embodied in many different forms and should not be construed as limited to the embodiments set forth herein; rather, these embodiments are provided for illustrative purposes only. Like numbers refer to like elements throughout.

While the embodiments of the suction valve and method for using the suction valve for regulating the flow of air and water through a medical instrument are described below in the context of an endoscope for performing an endoscopic procedure (such as a colonoscopy), it should be understood that the embodiments of the present invention may also be utilized in other medical instruments including, for example, a variety of different endoscopic and/or laparoscopic instruments.

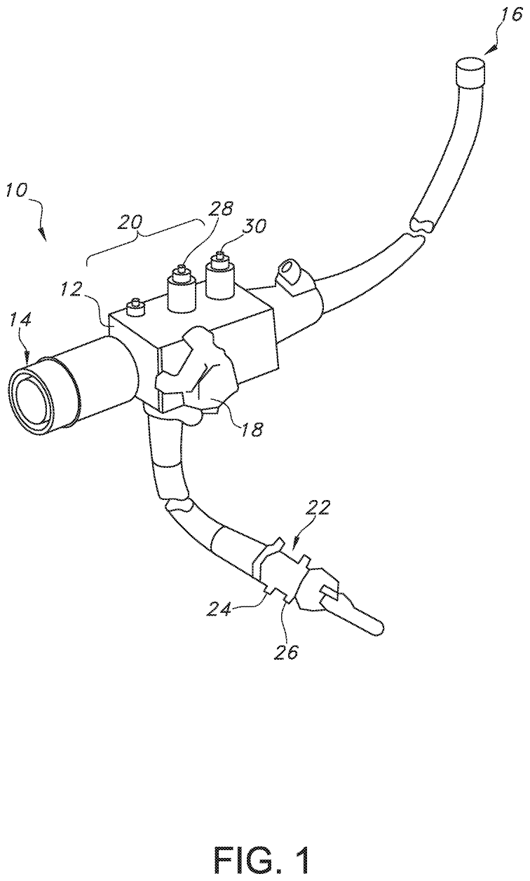

With reference to FIG. 1, an example of an endoscope 10 for performing endoscopic procedures, such as a gastrointestinal endoscopy, is shown. The endoscope 10 includes a proximal end 12, which may be the end closest to the practitioner or user and may include an eyepiece 14 that the user can look through to view the organ or cavity being examined. Alternatively, the endoscope 10 may include a CCD image sensor with the images being viewed on a monitor. The endoscope 10 also includes a flexible tube 15 with a distal end 16, which may be the end closest to the target site being examined. Controls 18 may be provided on a main body 20 of the endoscope disposed between the proximal and distal ends 12, 16. Furthermore, a fluid conducting portion 22 may be provided that connects a source of air and water (not shown) with the distal end 12 of the endoscope 10. For example, air, CO₂, or other gas for distending the organ or cavity is able to enter the endoscope 10 via a gas inlet 24, and water is able to enter via a water inlet 26. The flow of either fluid towards the distal end 20 may be regulated and controlled via the user's interaction with the controls of the main body 20.

A suction valve 28 is removably disposed within the main body 20 of the endoscope 10 to permit the user to withdraw effluent and gases from the patient, such as from the gastrointestinal tract. An air/water valve 30 may also be inserted in the main body 20 to permit a user to control the flow of air and/or water through the endoscope. The air water valve 30 may be of a type known in the art for selectively supplying air and water to the target site during a procedure.

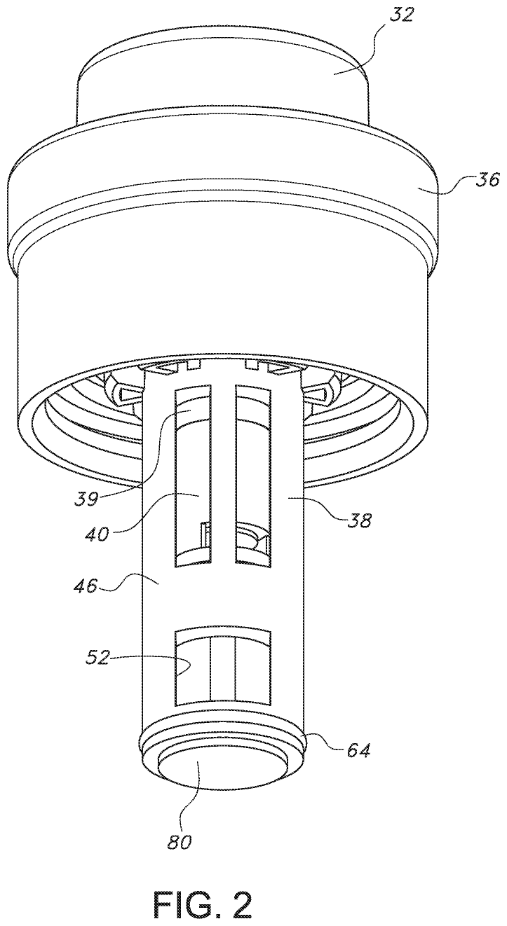

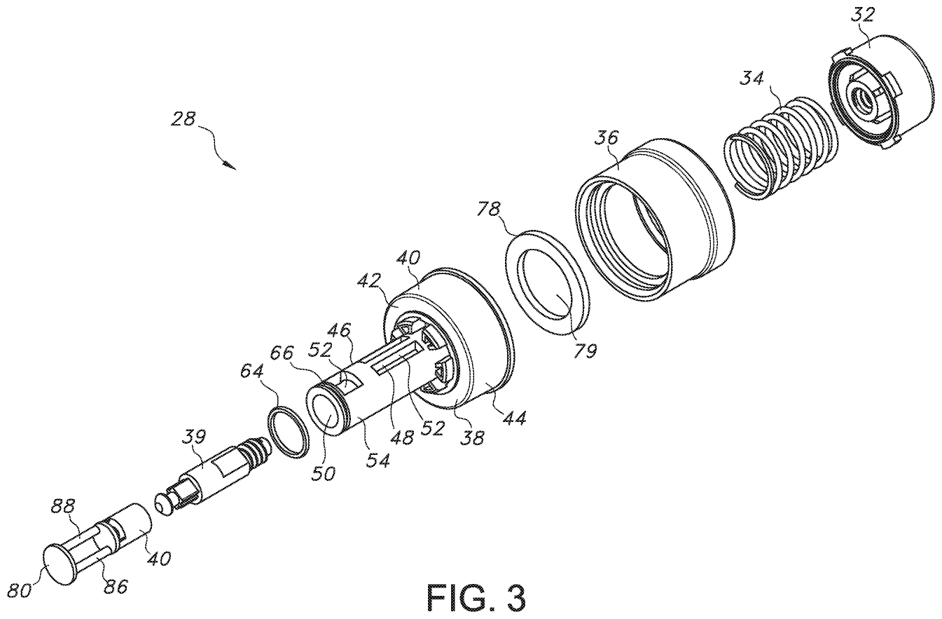

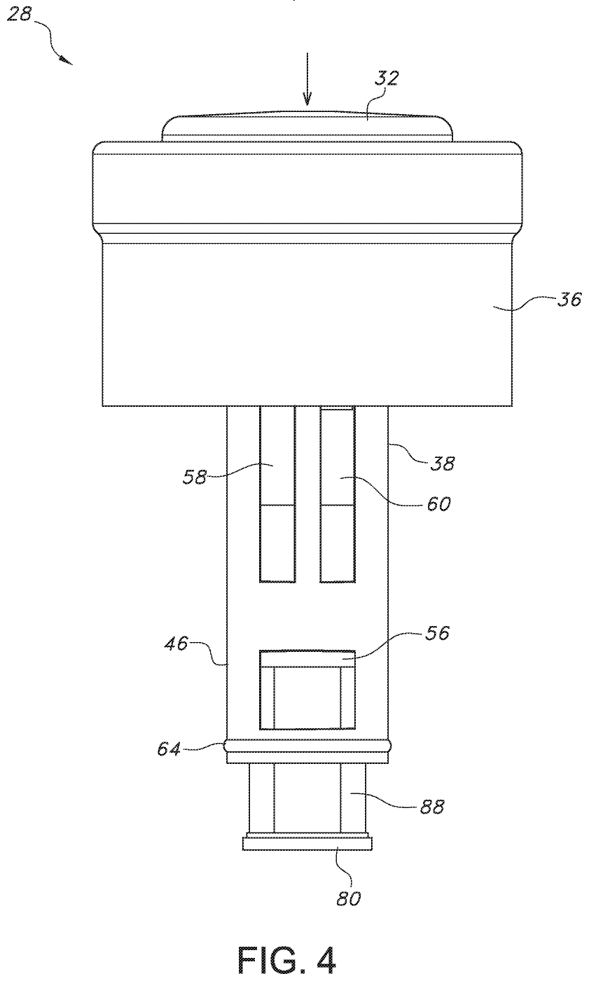

With reference to FIGS. 2-5, the suction valve 28 of the present disclosure may generally include an actuator 32, biasing device 34, a shell 36, valve body 38, stem 39, and sealing member 40. The valve 28 is removably insertable into the main body of endoscope.

The valve body 38 includes a cup-shaped upper end 40 having a base wall 42 perimetrically bounded by an upwardly extending sidewall 44. The base wall 42 has an annular opening operably connected to a cylindrical shaft 46 projecting from the cup-shaped upper end 40. The shaft 46 may be formed separately from the upper end 40 and then connected thereto by a snap-fit or other known connection method. Alternatively, the shaft 46 and upper end 40 may be formed as one integral element. The shaft 46 defines a valve body interior 48 extending between an open distal end 50 and a base opening 51 disposed in the base wall 42. The shaft 46 may include a plurality of apertures 52 formed in a shaft wall 54 therein to permit the flow of air, fluid and material therethrough. The apertures 52 permit biopsied tissue samples to pass through. In one embodiment, the plurality of apertures 52 include a first rectangular shaped aperture 56 disposed below a second 58 and third 60 more narrow rectangular-shaped apertures. The base wall 42 also have openings therein 62 to permit the flow of outside or ambient air. The shaft 46 adjacent its distal end may include a sealing element 64 in the form of an O-ring. An annular groove 66 may be formed in the shaft sidewall in order to retain the sealing element 64.

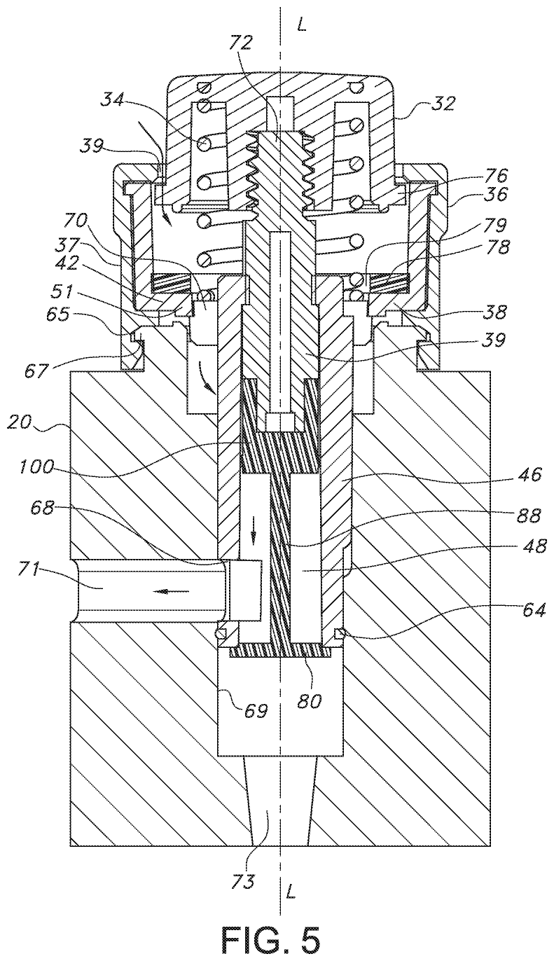

With specific reference to FIG. 5, the endoscope main body 20 includes a cylindrical channel 68 in which the shaft 46 of the valve body is inserted. The endoscope channel 68 communicates with the insertion tube 15 having via a tube port 73. The channel 68 is also in communication with a suction pump (not shown) via a suction port 71 which provides a negative pressure for generating a suction flow. The valve 28 is removably secured in the endoscope body 20 by the shell 36. The shell 36 is selectively attachable and removable from the endoscope. The shell 36 is in the form of an annular member having a perimeter wall 37 and an opening 39. The opening 39 permits the actuator to extend therethrough. A connection end 65 of the perimeter wall joins with cooperating structure 67 on the endoscope body. The connection may be in the form of a cooperating groove and protruding rim as shown in FIG. 5.

Alternatively, the shell 36 could be joined to the endoscope body by a threaded connection (not shown) or other connection manner know in the art. Once installed in the endoscope, the entire valve body 38 remains stationary relative to the main body 20 during the opening and closing of the suction valve 28. The sealing element 64 sealingly engages the channel sidewall 69. Therefore, when the suction valve is open, flow from the endoscope distal end 16 is directed through the valve body interior 48. The suction valve 28 selectively opens and closes the communication between the patient side and the suction pump as will be described in detail below.

The valve stem 39 is joined to the sealing member 40 and are both are translationally disposed within the valve body interior 48. The stem 39 may be formed of a rigid material and extends upwardly from the valve body interior into a chamber 70 created by the sidewall 44 and base wall 42. The stem upper end 72 is secured to and engages the actuator 32. In one embodiment, the stem upper end 72 may be threading secured to the actuator 32. The biasing device 34 is disposed between the actuator 32 and the bottom wall 42.

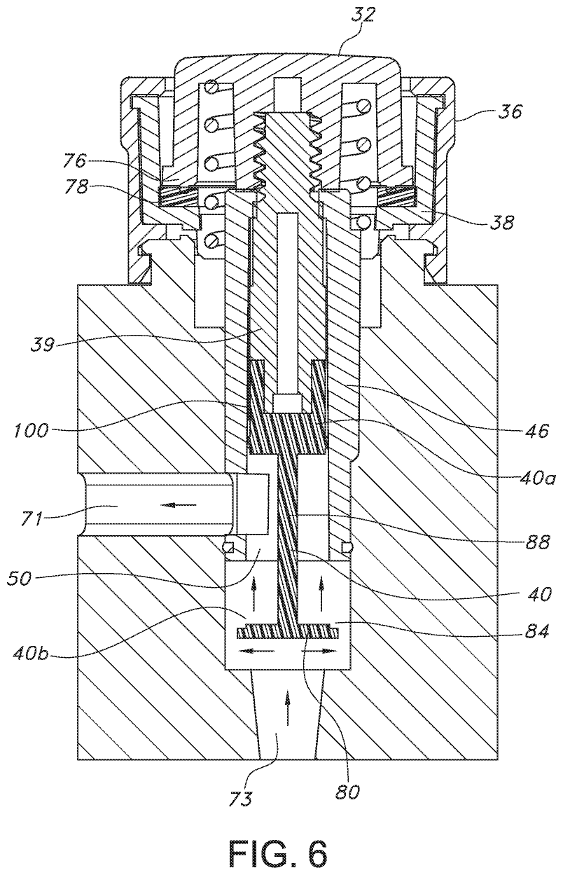

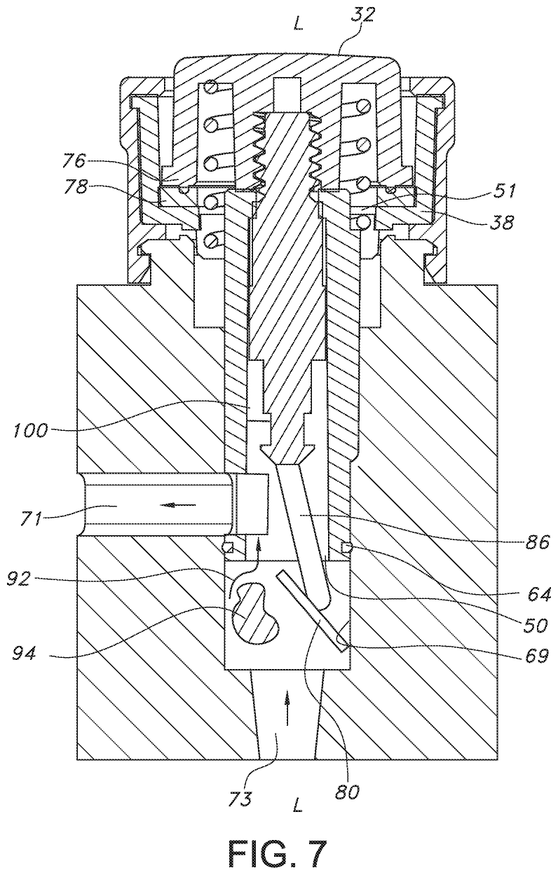

Depressing and releasing the actuator 32 selectively opens and closes the suction valve 28. Specifically, when the actuator 32 is depressed by the practitioner, the stem 39 and sealing member 40 are displaced from a valve closed position (FIG. 5) to a valve open position as shown for example in FIGS. 6 and 7. Releasing the actuator 32 causes the stem and sealing member 40 to return to the valve closed position as shown in FIGS. 2 and 5.

Movement of the actuator 32 between the open and closed position also causes a flow of ambient air to be controlled. The actuator 32 may have an extending annular flange 76 wider than the shell opening 39 such that the cap is retained in the shell even under the force of the biasing device 34. This flange 76 may also engage an annular gasket 78 disposed in the valve body upper end 40 on the base wall 42. The gasket 78 has a central opening 79 to permit the stem 39 and biasing device 34 to extend therethrough. As shown in FIGS. 6 and 7, when the valve 28 is actuated to the open position, the actuator 32 is depressed downwardly until the flange 76 engages the gasket 78. The engagement seals off the passage of ambient air traveling around the actuator cap 32 and valve body.

The sealing member 40 is formed of a flexible, deformable material such as silicon or silicone rubber, TPE (thermoplastic elastomer), TPR (thermoplastic rubber), or PVC. The sealing member 40 has a proximal end 40a fixedly secured to the stem 39 and an opposed distal end 40b forming a valve seal 80. The valve seal 80 has a central portion 82 bounded by an outwardly extending lip 84, the lip having a thickness less than the central portion. The sealing member distal and proximal ends may be joined by a flexible connecting member 86. The connecting member may include one or more spaced elongate members 88. The members 88 may be relatively thin compared to their length. The members 88 are flexible such that they permit the valve seal 80 to angularly deflect with respect to the longitudinal axis of the shaft L-L as shown in FIG. 7.

With reference to FIG. 5, the valve seal 80 is in sealing engagement with the valve body open distal end 50 when the suction valve 28 is in the closed or unactuated position. In this position the sealing member 40 and stem 39 are aligned along the longitudinal axis of the shaft L-L. Thus, the valve seal 80 occludes the end of the valve body and prevents the suction flow from acting on the patient side of the endoscope. In this closed position, the negative pressure generated by the suction flow remains present at the suction pump port, however, ambient air flows past the actuator cap 32, through the valve body, and out of the suction port 71 as shown by arrows 90. This flow assists in removing any fluid or material from the inside the valve body and the gap 100 between the valve stem and valve member and the valve body. The O-ring seal 64 and the valve seal 80 prevent flow from the patient side port 73 of the endoscope. Therefore, no air or fluid is evacuated from the patient.

As shown in FIGS. 6 and 7, when the valve 28 is actuated to the open position the engagement between the actuator 32 and gasket 78 seals off the passage of ambient air traveling around the actuator cap 32 and valve body as noted above. The downward travel of the actuator 32 also causes the valve seal 80 to move such that it separates from the bottom of the valve body and uncovers the opening 50. This permits air and fluid to travel between the valve seal 80 and valve body end through the apertures and out through the suction port as shown by arrows 92. Solids 94, such as biopsied tissue, may also flow through the valve. The flexibility of the sealing member causes such solids to deflect the valve seal 80 and provide a passageway for the material to flow.

The flow of the material 94 under the force of the suction will cause the valve seal 80 and the connecting members 88 to angularly deflect out of alignment with the longitudinal axis of the shaft L-L and toward the endoscope bore wall 69. In addition, the flexibility of the valve seal 80 will permit it to angularly deform from its generally perpendicular orientation relative to the axis L-L planar configuration. Furthermore, the flow of the material past the valve seal may cause it to deform its shape such as a curved generally U-shaped configuration. These deformations result in the cross-sectional area occupied by the valve seal 80 to be effectively reduced thereby creating a larger passage. Thus, material can flow past the valve seal 80 and connecting members 88 in an unobstructed manner.

Since the valve seal 80 acting on the end of the valve body 50 prevents material from passing into the valve body when the valve is in the closed position, there is no need to create a sealing engagement between the inside walls of the valve body and the sealing member 40 or stem 39. This permits the existence of the gap 100 between the surfaces which reduces the likelihood of clogging, sticking, or binding of the valve that would prevent the proper actuation and sealing of the valve 28. As noted above, this gap 100 can be cleared of fluid and/or material by the suction flow when the valve is in the unactuated state. Therefore, the valve 28 of the present disclosure provides the ability to permit passage of solids through the valve as well as preventing valve failure due to sticking or clogging.

Given the teachings provided herein, one of ordinary skill in the art will be able to contemplate other implementations and applications of the techniques and disclosed embodiments. Although illustrative embodiments have been described herein with reference to the accompanying drawings, it is to be understood that illustrative embodiments are not limited to those precise embodiments, and that various other changes and modifications are made therein by one skilled in the art without departing from the scope of the appended claims.

What is claimed is:

1. A suction valve comprising:
a valve body having a first portion and second portion including a sidewall extending from the first portion along a valve body longitudinal axis defining a valve body interior, the second portion having an open end forming an inlet and the side wall having an outlet;
a stem disposed in the valve body interior and operably connected to a sealing member, the sealing member having a valve seal disposed thereon, the valve seal being movable between a closed position wherein the valve seal seals the inlet and an open position wherein the inlet is unsealed; and
the sealing member having a flexible member extending from the stem, wherein the sealing member is angularly deflectable relative to the valve body longitudinal axis when in the open position upon operation of a fluid flowing through the inlet and the outlet.

2. The suction valve as defined in claim 1, wherein the valve seal is a planar flexible member.

3. The suction valve as defined in claim 1, wherein the flexible member includes at least one elongate member.

4. The suction valve as defined in claim 3, wherein the flexible member includes a pair of spaced elongate members having an opening therebetween.

5. The suction valve as defined in claim 1, wherein the valve body remains stationary upon movement of the valve seal between the open and closed position.

6. The suction valve as defined in claim 1, wherein the valve body first portion includes a base wall perimetrically bounded by a sidewall extending therefrom, the base wall and sidewall forming a chamber, an actuator disposed within the chamber and being operably connected to the stem.

7. The suction valve as defined in claim 6, wherein a biasing device is disposed between the actuator and the valve body for urging the valve seal into the closed position.

8. The suction valve as defined in claim 6, wherein a gasket is disposed in the chamber about an opening in the base wall, and wherein the actuator sealing engages the gasket when moved to the closed position thereby restricting a flow of air through the valve body.

9. The suction valve as defined in claim 1, wherein the valve seal has a central portion bounded by an outwardly extending lip, the lip having a thickness less than the central portion.

10. The suction valve as defined in claim 1, wherein a seal is disposed about the second portion and adapted to seal between the valve body and an endoscope.

11. A suction valve insertable in an endoscopic device having a main body including a bore defined by a bore wall, the bore in communication with a suction pump and an insertion tube, the section valve being disposed in the bore, the suction valve comprising:
a valve body having a first portion and second portion including a sidewall extending from the first portion along a valve body longitudinal axis defining a chamber, the second portion having an open end forming an inlet and the side wall having an outlet;
a stem disposed in the valve body and operably connected to a sealing member, the sealing member having a valve seal disposed thereon, the valve seal being movable between a closed position wherein the valve seal seals the inlet and an open position wherein the inlet is unsealed; and
the sealing member having a flexible member extending from the stem, wherein the sealing member is deflectable relative to the valve body longitudinal axis toward the bore wall when in the open position upon operation of a fluid flowing through the inlet and the outlet.

12. The suction valve as defined in claim 11, wherein the valve seal is a planar flexible member.

13. The suction valve as defined in claim 12, wherein the flexible member includes at least one elongate member.

14. The suction valve as defined in claim 13, wherein the flexible member includes a pair of spaced elongate members having an opening therebetween.

15. The suction valve as defined in claim 11, wherein the valve body remains stationary relative to the endoscope main body upon movement of the valve seal between the open and closed position.

16. The suction valve as defined in claim 11, wherein the valve body first portion includes a base wall perimetrically bounded by a sidewall extending therefrom, the base wall and sidewall forming a chamber, an actuator disposed within the chamber and being operably connected to the stem.

17. The suction valve as defined in claim 16, wherein a biasing device is disposed between the actuator and the valve body for urging the valve seal into the closed position.

18. The suction valve as defined in claim 16, wherein a gasket is disposed in the chamber about an opening in the base wall, and wherein the actuator sealing engages the gasket when moved to the closed position thereby restricting a flow of air through the valve body.

19. The suction valve as defined in claim 11, wherein the valve seal has a central portion bounded by an outwardly extending lip, the lip having a thickness less than the central portion.

20. The suction valve as defined in claim 11, wherein a seal is disposed about the second portion and adapted to seal between the valve body and an endoscope.

21. The suction valve as defined in claim 11, wherein a shell removably retains the suction valve in the endoscope main body.

22. A method of controlling suction of an endoscope comprising:
obtaining an endoscope including a suction valve, the suction valve including a valve body having a first portion and second portion including a sidewall extending from the first portion along a valve body longitudinal axis defining a chamber, the second portion having an open end forming a inlet and the side wall having an outlet, a stem disposed in the valve body and operably connected to a sealing member, the sealing member having a valve seal disposed thereon, the valve seal being movable between a closed position wherein the valve seal seals the inlet and an open position wherein the inlet is unsealed, an actuator operable connected to the stem; and the sealing member having a flexible member extending from the stem, wherein the sealing member is deflectable relative to the valve body longitudinal axis toward the bore wall when in the open position upon operation of a fluid flowing through the inlet and the outlet; and
actuating the actuator to cause the sealing member to move the open position to permit fluid to flow through the suction valve; and
deactivating the actuator to cause the sealing member to return to the closed position to restrict a flow of fluid through the suction valve.
